(19) United States
(12) Patent Application Publication
(10) Pub. No.: US 2022/0403389 A1
(43) Pub. Date: Dec. 22, 2022

(54) THERAPEUTIC OLIGONUCLEOTIDE METHODS

(71) Applicant: Q-STATE BIOSCIENCES, INC., Cambridge, MA (US)

(72) Inventors: Caitlin Lewarch, Cambridge, MA (US); David Gerber, Cambridge, MA (US); Luis Williams, Cambridge, MA (US); Duncan Brown, Cambridge, MA (US); Sudhir Agrawal, Cambridge, MA (US); Graham T. Dempsey, Sudbury, MA (US)

(21) Appl. No.: 17/842,352

(22) Filed: Jun. 16, 2022

Related U.S. Application Data
(60) Provisional application No. 63/211,814, filed on Jun. 17, 2021.

(51) Int. Cl. C12N 15/113; A61P 25/00; G16B 35/00

(52) U.S. Cl. CPC C12N 15/113; A61P 25/00; G16B 35/00; C12N 2310/341

(57) ABSTRACT

The invention provides systems and methods for discovering candidate therapies for genetic conditions and also for screening those therapies in vitro for evidence of neurotoxicity. Where a medical condition is a consequence of a genetic target such as a mutated gene, the disclosure provides in silico methods to generate lists of candidate sequences for antisense oligonucleotides (ASOs) that will potentially bind to the gene or transcripts from the gene in vivo and treat the associated condition by restoring a healthy phenotype of gene expression. The invention provides in vitro methods for screening candidate ASO sequences for symptoms of neurotoxicity in vivo. For example, candidate sequences that are output by the in silico analytical pipeline can be synthesized and assayed against live cells in vitro.

### TECHNICAL FIELD

The disclosure relates to therapeutic discovery.

### BACKGROUND

Epilepsy is an example of a neurological condition with unfortunate effects. People who suffer from epilepsy suffer from debilitating seizures and may experience abnormal sensations. It is thought that many cases of epilepsy have a genetic cause. Similarly, Parkinson's, Alzheimer's disease, and amyotrophic lateral sclerosis may also have genetic causes. Unfortunately, medical researchers have not in every case identified a drug that treats the disease.

In fact, drug discovery for such conditions may be stymied by a diversity of molecular mechanisms implicated in any given condition. For example, some people suffer from Angelman syndrome, which is associated with mutations that inactivate a gene for a ubiquitin ligase protein. There can be different abnormalities—mutations or rearrangements—in the region of chromosome 15 that contain the gene for that ligase. Children born with such mutations may exhibit delayed development, speech impairment, and problems with movement and balance.

Traditional drug discovery often involves screening for a small molecule that blocks or corrects a mis-functioning molecular target. However, with some categories of neurological conditions, there is not an evident molecular target for treatment with a small molecule drug. In another example, Hutchinson-Gilford syndrome is a condition that manifests as the rapid appearance of aging in childhood. It is thought that this condition is caused by a mutation in a gene that causes RNA transcribed from that gene to be mis-assembled. Again, there is no self-evident target for a traditional small-molecule drug. Accordingly, a variety of medical conditions are difficult to treat and continue to have unfortunate effects on people's lives.

### SUMMARY

The invention provides systems and methods for discovering candidate therapies for genetic conditions and also for screening those therapies in vitro for evidence of neurotoxicity. Where a medical condition is a consequence of a genetic target, such as a mutated gene, the invention provides in silico methods to generate lists of candidate sequences for antisense oligonucleotides (ASOs) that will potentially bind to the gene or transcripts from the gene in vivo and treat the associated condition by restoring a healthy phenotype of gene expression. For example, the ASOs can be designed as DNA-containing gapmers that hybridize to disfavored transcripts and mark those for degradation by RNAse. Alternatively, the ASOs can be designed to bind to pre-RNA and mask disfavored splice sites and promote splicing of the preRNA into a healthy isoform of the mRNA. In another example, the ASO can be designed to hybridize to a target and sterically block the binding of other molecules, such as miRNAs that may inhibit translation of an mRNA, thereby rescuing a healthy phenotype. Methods of the invention embody rules important for design of several categories of ASOs in software packages in silico. Systems of the invention can query a gene sequence (e.g., from GenBank) and generate every possible ASO sequence meeting certain criteria and also further apply analytical modules to identify, or rule out, sequences that are predicted to exhibit undesired binding behaviors such as poor change in Gibbs free energy on binding to target, or preferential binding to non-target or to self. Modules of the invention can evaluate candidate ASO sequences for target accessibility, e.g., to identify targets with competing oligonucleotide or protein binding. The resultant output is a set of candidate ASO sequences with potential clinical utility. A non-limiting set of targets include SCN8A, SCN9A, SCN10A, UBE3A, STXBP1, and SYNGAP1.

The invention then provides in vitro methods for screening candidate ASO sequences for symptoms of neurotoxicity in vivo. For example, candidate sequences that are output by the in silico analytical pipeline can be synthesized and assayed against live cells in vitro. Those live cells may be neurons, such as induced-Pluripotent Stem Cell (iPSC)-derived neurons grown in vitro with optogenetic constructs that allow for optical, e.g., fluorescent, recording of neuronal activity using, for example, microscopes or optical sensors and analytical systems. In fact, the analytical system can record firing patterns such as spike frequency and action potential waveform shape of live neurons treated with the oligonucleotides proposed by the in silico platform. The analytical system may extract significant features from those firing patterns to, for example, candidate ASO therapies that rescue healthy phenotypes in neurons with disease-associated genotypes. Also, of significance, the analytical system can (independently of predicting treatment efficacy) detect cautionary symptoms of neurotoxicity when neurons in vitro are exposed to ASO sequences generated in silico. The in vitro neurons can live in wells of, e.g., 96-, 384-, 1536-, or 3456-well plates, and can be exposed to a large and diverse pool of candidate ASOs rapidly and in parallel. Independently of looking for phenotype rescue, the analytical system can capture patterns of neuronal activity induced by ASO exposure and detect features predictive of neurotoxicity in those patters. The analytical systems may include large data stores created by high-throughput screening over time, e.g., over neurons and drugs with known effects. In fact, the analytical system may include machine learning systems trained on the data store with the known effects. The machine learning systems (e.g., neural networks, random forests, support vector machines, others, or combinations thereof), may read patterns of neuronal activity arising from exposure to one of the newly synthesized ASOs output by the in silico pipeline and given an output rating the ASO for neurotoxicity.

Both the in silico and the in vitro platforms can be automated. The entire process can begin through software modules implemented in server or cloud computing environments. The candidate ASO sequences can be passed to an oligonucleotide synthesis platform or service. Synthetic oligonucleotides can be handled, e.g., by liquid handling robots, for exposure to the optogenetic iPSC-derived neurons in wells of the multiwell plates. Plate imaging can be performed by an automated fluorescent plate reader or microscope and analytical systems such as machine learning system can read and detect neurotoxicity predictive features from neuronal activity (e.g., spike frequency or action potential shape) read from the wells. Thus, the invention provides an integrated pipeline and platform for the design and toxicity pre-screening for therapeutic compositions for genetic conditions.

In one exemplary embodiment, ASO design begins with selection of a gene implicated in a condition. The design involves the generation of all possible antisense N-mers (e.g., ASOs which are 20 nucleotides in length) targeting an mRNA or pre-mRNA transcript, though the pipeline is flexible in terms of oligonucleotide length. For each of these candidate sequences, the in silico platform evaluates a variety of sequence characteristics, including thermodynamic parameters that reflect its binding to its intended target or to itself, and its sequence matches to unintended targets in the human and optionally one or more non-human model organisms. ASOs that emerge from the in silico design platform are screened in vitro for evidence of in vivo neurotoxicity. The in vitro screening may use “Optopatch”, a combination of microbial proteins that, in iPSC-derived neurons, optically recapitulates patch clamp technology, albeit in a high-throughput manner. Optopatch proteins generate optical signals that are used to record activity patterns for the neurons, which patterns are analyzed for evidence of neurotoxicity.

In certain aspects, the invention provides methods that include generating a list of oligonucleotide sequences that are substantially complementary to a genetic target implicated in a disorder; analyzing the sequences via in silico operations that remove sequences according to pre-determined criteria, leaving a filtered list; obtaining oligonucleotides made with sequences from the filtered list; and exposing one or more live cells to the oligonucleotides in vitro to identify candidate therapeutic oligonucleotides that do not induce an adverse phenotype in the live cells. The genetic target may be a gene and the list of oligonucleotide sequences may be a list of substantially every N-mer complementary to a subsequence of the gene (e.g., for 15<N<25). The in silico operations may include comparing each oligonucleotide sequence to a genome and removing ones that are substantially complementary to a sub-sequence in the genome outside of the genetic target. The in silico operations may include removing sequences from the list for which a Gibbs free energy change for binding to target is insufficiently favorable. The in silico operations may include a software module that models duplex formation and associated Gibbs free energy changes to exclude sequences that: form dimers, form hairpins, or bind off-target. In some embodiments, the in silico operations include comparing the list of oligonucleotide sequences or the genetic target to a genome of a non-human model organism (e.g., primate or rodent) to identify a genetic target with homologous target in the non-human model organism.

Transitioning from the in silico to the in vitro components may involve nucleotide synthesis. The obtaining step may include ordering and receiving synthetic oligonucleotides for each of the sequences from the filtered list. Exposing the live cells to the oligonucleotides in vitro may include performing all-optical electrophysiology or Optopatch to obtain a neural phenotype for the cells when exposed to the oligonucleotides.

For the in vitro exposing step, the live cells may include e stem-cell derived neurons in vitro. In certain embodiments, at least one of the neurons expresses an optical reporter of membrane potential (e.g., such as an optionally-modified microbial rhodopsin). The method may include using a light detector or sensor to read a neural activity phenotype of the neuron when exposed at least one of the oligonucleotides. The neurons may include a light-gated ion channel e.g., as an optical actuator of neural firing (suitable channels may include optionally-modified version of an algal channelrhodopsin such as CheRiff). The neural activity phenotype may be analyzed against a data store (e.g., terabytes or petabytes of historical Optopatch recordings) of phenotypes. The analysis may be performed by a machine learning system trained on the data store, with phenotypes in the data store being associated with labels, such as for condition or toxicity. In some embodiments, phenotypes in the data store are labeled by neurological conditions that include one or more of epilepsy, autism, movement disorders, developmental delay disorders, arthritis, chronic pain, and Alzheimer's disease. The method may include operating a machine learning system to detect phenotypes associated with oligonucleotide toxicity.

In gapmer embodiments, the in silico operations include predicting the performance of the oligonucleotide sequences as gapmers that will mediate enzymatic degradation of an RNA. The genetic target may be, for example, a gene for a sodium channel and the disorder is chronic pain associated with cancer or arthritis. In splice-modulating embodiments, the in silico operations include predicting the performance of the oligonucleotide sequences splice-modulating oligonucleotides that promote splicing of a pre-RNA to form a preferred isoform of an RNA. In steric blocking embodiments, he in silico operations include predicting the performance of the oligonucleotide sequences as steric blocking oligonucleotides that inhibit the function of a micro-RNA.

The in silico operations may include presenting the oligonucleotide sequences to a predictive module that predicts target-binding by comparison to results from transcriptomic analysis assays performed with test oligonucleotides. The predictive module may use a machine learning system to predict expression modulation of off-target genes for each oligonucleotide sequence, the machine learning system trained on results of expression analysis for a plurality of antisense oligonucleotides. Preferably the in silico operations include the application of sequence distance rules to avoid off-target effects, wherein the rules exclude sequences for which the genome includes a non-target region that aligns to the sequence with an exact match, mismatch, or at least a threshold number of consecutive matches. The in silico operations include a software package that performs a pairwise alignment of each of the oligonucleotide sequences to a human genome or to a primary transcript sequence for a gene that includes the genetic target to exclude sequences with off-target binding affinity. Optionally, the in silico operations include evaluating, for each oligonucleotide sequence, accessibility of a binding site in the genetic target. Accessibility may be evaluated by a software module that predicts secondary structure or binding protein occupancy in an RNA transcript of the genetic target.

In some embodiments, the genetic target is a gene. The list of oligonucleotide sequences may be generated by a software module that queries a genetic database for a gene sequence of the gene and parses the gene sequence to generate the list. Optionally the in silico operations are performed automatically by a computer system that outputs the filtered list (e.g., as a FAST file) as an order form for an oligonucleotide synthesis service. The exposing step may involve transfer by liquid handling systems of synthetic oligonucleotides into wells of multiwell plates that include the live cells, wherein the live cells are neurons, wherein at least one neuron expresses a microbial rhodopsin that functions as an optical reporter of membrane potential in the neuron.

Aspects of the disclosure provide a method of detecting toxic effects of a composition. The method includes obtaining a composition that interacts with a genetic target to affect neural function; measuring activity of a neuron exposed to the composition in vitro; and detecting, in the activity measurements, features that are predictive of in vivo toxicity of the composition. The composition may include an antisense oligonucleotide that hybridizes to the genetic target. Preferably the measured activity includes an action potential waveform or spike train of action potentials of the neuron. The features predictive of in vivo toxicity may include hyper- or hypo-excitability of the neuron. The neuron may express a microbial rhodopsin that optically reports membrane electrical potential (e.g., Arch D95N or a QuasAr). Additionally or alternatively, the neuron may express at least one of a light-gated ion channel (e.g., CheRiff) and/or genetically-encoded calcium indicator (e.g., a gCaMP protein).

The detecting step may include comparing the measured activity to control activity measured from one or more neurons not exposed to the composition. In some embodiments the detecting step is performed by a machine learning system trained on training data comprising measurements from a plurality of neuronal samples made under known conditions. The detecting step may be performed by a machine learning implemented in a computer system. The predictive features may be detected by a system trained to detect features known to indicative of neurotoxicity.

### BRIEF DESCRIPTION OF THE DRAWINGS

FIG. 1 diagrams a method of the invention.

FIG. 2 shows in silico operations.

FIG. 3 shows success of the ASO design tools.

FIG. 4 shows modulation in vitro of unintended targets.

FIG. 5 shows predicted thermodynamic properties of ASO binding.

FIG. 6 shows all-optical electrophysiology with Optopatch.

FIG. 7 shows membrane trafficking of CheRiff in a rat hippocampal neuron.

FIG. 8 shows QuasAr fluorescence.

FIG. 9 shows a CAD model of a 96-well microscope.

FIG. 10 shows the light path for coupling red laser light into cell samples.

FIG. 11 gives example voltage recordings.

FIG. 12 gives a Raster plot of results.

FIG. 13 shows the spike rate averaged over cells.

FIG. 14 shows spike shape recorded by the optogenetics microscope.

FIG. 15 gives spike timing properties (e.g., firing frequency).

FIG. 16 illustrates that adaptation are automatically extracted for each cell.

FIG. 17 shows the excitability.

FIG. 18 is a Raster plot from the ALS-KD results.

FIG. 19 shows firing rate average in the ALS-KD embodiment.

FIG. 20 is a spike waveform from the ALS-KD embodiment.

FIG. 21 shows qPCR validation of ALS-KD target.

FIG. 22 shows protein knockout in a disease model.

FIG. 23 shows phenotypes for various cells treated with drugs targeting the pathway downstream of the target gene.

FIG. 24 shows results from heterozygous patient cell lines and healthy familial controls.

FIG. 25 gives a multidimensional radar plot.

FIG. 26 shows the results of a large screen of a compound library.

FIG. 27 shows drug similarity comparisons.

FIG. 28 show results from rat hippocampal cultures treated with ASOs.

FIG. 29 show results when ASOs and vehicle were intrathecally delivered.

FIG. 30 shows a UMAP projection of >600 intrinsic excitability features for ASO-treated neurons

### DETAILED DESCRIPTION

The invention provides an integrated platform for the design and discovery of therapeutic antisense oligonucleotides for CNS diseases. The platform implements a method that includes generating a list of oligonucleotide sequences that are substantially complementary to a genetic target implicated in a disorder; analyzing the sequences via in silico operations that remove sequences according to pre-determined criteria, leaving a filtered list; obtaining oligonucleotides made with sequences from the filtered list; exposing one or more live cells to the oligonucleotides in vitro to identify candidate therapeutic oligonucleotides that do not induce an adverse phenotype in the live cells. Antisense oligonucleotides (ASOs) are tools to modulate gene expression and have emerged as an approach to the treatment of devastating disorders of the nervous system. ASOs have now demonstrated clinical success in the treatment of Spinal Muscular Atrophy, with potential use for treating severe neurological disorders such as Dravet syndrome, ALS, Huntington's Disease and Angelman syndrome.

Systems and methods of the invention are useful to effectively design ASOs without toxic liabilities in the CNS. Using methods of the disclosure, the relative binding affinities of ASOs to both intended and unintended RNA targets are predicted in silico. The systems and methods comprehensively evaluate such predictions for the most clinically-relevant chemistries and lengths. Systems and methods of the disclosure test ASO activity in neurons to predict effects in the central nervous system (CNS). As preclinical in vivo toxicity studies are expensive and generally limited to a small number of ASO candidates, the development of tools which would identify ASOs with neurotoxic effects prior to in vivo studies will be helpful in identifying therapeutics useful to treat people for problematic conditions.

The invention provides tools to design ASOs that modulate the level of therapeutically relevant RNAs in neurons. Systems and methods of the invention integrate neuronal-based disease models, high-throughput all-optical electrophysiology (or Optopatch), and machine-learning based analytics. Methods of the invention systematically characterize the sequence and thermodynamic rules that govern ASO modulation of unintended RNA targets. Those methods are useful to demonstrate that in vivo toxic neurological properties can be predicted in vitro using the Optopatch platform. Methods may be used, for example, for designing and comprehensively evaluating ASOs to modulate target genes such as UBE3A and SHANK3. Systems and methods of the disclosure are useful to identify neurotoxic ASOs early in the design and screening process, thereby accelerating our development of novel CNS therapeutics.

Systems and methods of the disclosure are useful to systematically characterize the relationship between sequence distance and off-target ASO activity for both gapmers and steric blocking oligonucleotides. Sequence complementary to an intended target and sequence distance (number of mismatches) from unintended targets drive ASO specificity, but the distance thresholds that determine interactions with unintended targets are not clearly defined for the most clinically relevant oligonucleotide chemistries and lengths. Systems and methods of the disclosure are useful to identify the sequence similarity thresholds that predict the off-target activity of 2′-MOE-containing gapmers (which promote RNase H-mediated decay of their targets) and steric blocking oligonucleotides (which modulate splicing, stability, or downstream translation) using various strategies. For example, systems and methods of the disclosure are useful to generate variants of known effective ASOs and characterize their on-target efficacy to probe the effects of sequence substitutions on activity. Some embodiments use bulk RNA-seq to evaluate transcriptome-wide gene expression and isoform composition in cultured neurons treated with a panel of informative ASOs to identify off-target activity at various levels of sequence similarity.

Systems and methods of the disclosure are useful to make in vitro functional measurements of neurons that predict in vivo CNS toxicity. A platform such as Optopatch may be used to characterize the behavior of rodent primary and human iPSC-derived neurons treated with ASOs known to be neurotoxic in vivo and a large panel of diverse, non-targeting ASOs.

Antisense oligonucleotides (ASOs) are useful tools to modulate gene expression and have emerged as an approach to the treatment of devastating disorders of the nervous system. ASOs as a therapeutic modality have several unique intrinsic advantages. Sequence complementarity allows an ASO to precisely bind to and modulate the levels of its RNA target, while transcripts with nucleotide mismatches relative to the intended target (or those with sufficient sequence distance from that intended target) are spared. Specific chemical modifications stabilize ASOs and allow them to downregulate target gene expression via RNase H-mediated decay (ASOs synthesized with “gapmer” chemistry) or bind to a target transcript in sites that modulate splicing, stability, or downstream translation (ASOs synthesized with RNA-like “steric blocking” chemistry). Mechanisms of gene regulation by ASOs potentially include (i) RNaseH-mediated degradation towards gene knockdown and (ii) splice modulation towards restoring gene expression.

The invention addresses genetic diseases that can be specifically addressed by ASOs designed to correct dysregulated mRNA or protein levels at their root cause. ASOs have potential for treating severe disorders of the central nervous system (CNS) including rare diseases such as Dravet Syndrome, ALS and Angelman Syndrome. Importantly, using systems and methods of the invention, the timeline from project inception to clinical trials for ASO-based therapeutics is much shorter than the timeline for more traditional small molecule-based therapeutics.

Despite the emerging success of ASO-based medicines, there remains significant clinical and commercial opportunity to identify and avoid ASOs with toxic liabilities, e.g., those with neurotoxic effects. While the relative binding affinities of ASOs to both on- and off-target transcripts can be predicted in silico based on sequence alone using methods of the invention, publicly disclosed heuristics used to exclude off-target binding sites are often poorly supported by transcriptome-wide evidence or are based on the properties of ASOs synthesized using chemistries that have not progressed clinically, such as gapmers containing locked nucleic acid (LNA) bases. Furthermore, in vivo toxicity that is not related to the direct Watson-Crick binding of the ASO to an RNA target is more difficult to predict based on sequence. Many of the assays and tools developed to predict or screen for toxic ASOs have used LNA-based libraries or specifically examined liver and blood toxicity resulting from systemic administration of the candidate therapeutics or their delivery to cultures of non-CNS cell types. Those platforms do not reveal effects of ASOs in neurons of the CNS. The invention provides a high-throughput, information-rich in vitro screening assay in human neurons to accelerate and improve the design of effective, non-toxic ASO therapeutics for disorders of the nervous system.

The in silico operations may include software modules that embody sequence rules governing off-target effects mediated by Watson-Crick base pairing for the most clinically relevant ASO chemistries. Those rules may be obtained from experiments such as transcriptomic analysis and systematic examination of a large number of variants of active ASOs. Methods of the invention include an in vitro screening assay using functional measurements of human cellular models to evaluate potential toxicity in the CNS. Those assays may harness the capabilities of all-optical electrophysiology such as the Optopatch platform. Optopatch provides electrophysiological investigations of neurons with the information content of manual patch clamp, but with >10,000-fold higher throughput. Embodiments of the invention combine that determinative measurement platform with machine learning-based analytics and advanced genetic disease models to create an integrated technology platform for drug discovery in CNS-based disorders.

Embodiments provide a method of detecting toxic effects of a composition. The method includes obtaining a composition that interacts with a genetic target to affect neural function; measuring activity of a neuron exposed to the composition in vitro; and detecting, in the activity measurements, features that are predictive of in vivo toxicity of the composition. The composition includes an antisense oligonucleotide (ASO) that hybridizes to the genetic target. The neurons express optical reporters of membrane potential (e.g., an optionally-modified version of Archaerhodopsin 3 such as Arch3 D95N or a QuasAr protein), used for reading and recording an action potential waveform or spike train of action potentials of the neuron. An analytical system such as a machine learning system detects phenotypes associated with oligonucleotide toxicity. The machine learning system may be trained on training data comprising measurements from a plurality of neuronal samples made under known conditions. Thus, the invention provides a technology platform with the ability to measure perturbed neuronal physiology as a predictive readout of neurotoxicity.

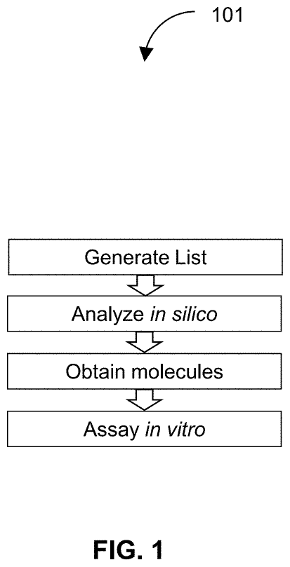

FIG. 1 diagrams a method 101 that includes generating a list of oligonucleotide sequences that are substantially complementary to a genetic target implicated in a disorder; analyzing the sequences via in silico operations that remove sequences according to pre-determined criteria, leaving a filtered list; obtaining oligonucleotides made with sequences from the filtered list; and exposing one or more live cells to the oligonucleotides in vitro to identify candidate therapeutic oligonucleotides that do not induce an adverse phenotype in the live cells. The method uses computer and laboratory systems that embody and provide pipelines to design ASOs.

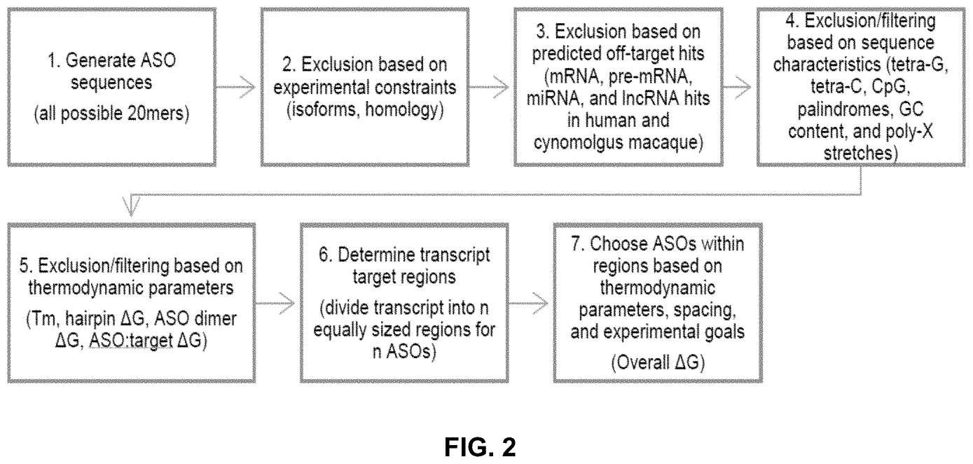

FIG. 2 shows in silico operations that may be included in an ASO Design Pipeline. Briefly, ASO design begins with the generation of all possible antisense 20 mers (ASOs which are 20 nucleotides in length) targeting an mRNA or pre-mRNA transcript, though the pipeline is flexible in terms of oligonucleotide length. Preferably the list of oligonucleotide sequences is generated (1) by a software module that queries a genetic database for a gene sequence of the gene and parses the gene sequence to generate the list. Certain experimental constraints may be used to exclude (2) certain sequences in one of the in silico operations. For example, sequences lacking homologous targets in a model organism or those that do not bind to all transcript isoforms of the target gene may be excluded (2). The in silico operations include comparing each oligonucleotide sequence to a genome and removing (3) “off-target hits”, i.e., ones that are substantially complementary to a sub-sequence in the genome outside of the genetic target. It may be beneficial for the operations to exclude or filter (4) based on sequence liabilities such as tetra-C or G, CpG islands, palindromes, GC content, long polypurine stretches, long polypyrimidine stretches, or homopolymer runs. Further, the in silico operations may include removing (5) sequences from the list that are prone to hairpin or dimer formation or have insufficient binding affinity to their intended target. The operations may model target accessibility and select for inclusion, i.e., exclude (6) certain sequences to ensure good coverage across a target. The in silico operations may present oligonucleotide sequences to a predictive module that predicts target-binding by comparison to results from transcriptomic analysis assays performed with test oligonucleotides. Additionally or in combination, the in silico operations may include a software module that models duplex formation and associated Gibbs free energy changes to exclude (7) sequences that: form dimers, form hairpins, or bind off-target. Preferably, these in silico operations are performed automatically by a computer system that outputs the filtered list as an order form for an oligonucleotide synthesis service.

The in silico operations aid in selecting sequences for ASOs. The in silico operations may predict the performance of the oligonucleotide sequences as gapmers that will mediate enzymatic degradation of an RNA.

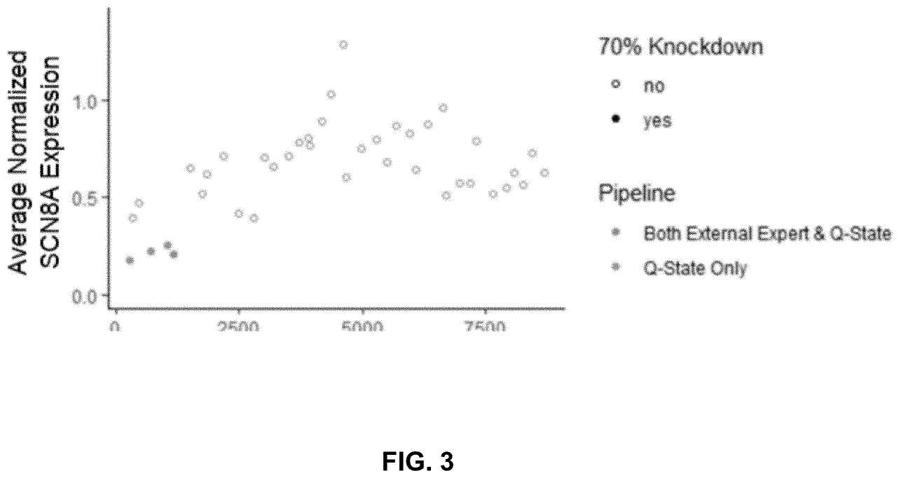

FIG. 3 shows knockdown of SCN8A in an example where the genetic target is a gene for a sodium channel and the disorder is chronic pain associated with cancer or arthritis.

Optionally, the in silico operations include predicting the performance of the oligonucleotide sequences splice-modulating oligonucleotides that promote splicing of a pre-RNA to form a preferred isoform of an RNA. Additionally or alternatively, the in silico operations may include predicting the performance of the oligonucleotide sequences as steric blocking oligonucleotides that inhibit the function of a micro-RNA.

ASO candidates are filtered to adhere to experimental requirements, avoid sequence liabilities, and ensure favorable thermodynamic properties. The transcript is preferably tiled to identify accessible regions. The in silico operations may include the application of sequence distance rules to avoid off-target effects, wherein the rules exclude sequences for which the genome includes a non-target region that aligns to the sequence with an exact match, mismatch, or at least a threshold number of consecutive matches.

For each of the candidate sequences, a variety of sequence characteristics are evaluated, including thermodynamic parameters that reflect its binding to its intended target or to itself, and its sequence matches to unintended targets in the human and cynomolgus macaque transcriptomes. Given the need for downstream in vivo toxicology studies in non-human primates as part of any ASO pre-clinical development plan, it may be preferable require exact homology in a non-human primate (e.g., cynomolgus macaque) and, depending on the biology of the gene and availability of animal models, optionally in other species or matches in a certain number of transcript isoforms in humans (FIG. 2, step 2). I.e., the in silico operations include comparing the list of oligonucleotide sequences or the genetic target to a genome of a non-human model organism to identify a genetic target with homologous target in the non-human model organism. In steps 3 through 5 of exemplary embodiments, the thousands of candidate sequences are then filtered using a series of thresholds to prioritize candidates with favorable binding properties and without sequence liabilities or close sequence alignments to unintended transcripts. That filtered pool generally contains hundreds of ASOs with favorable properties distributed across the target. Target accessibility is a major factor in determining ASO efficacy, and step 6 identifies accessible regions empirically by tiling the transcript as broadly as possible. To identify the most promising ASOs, at step 7 regional representatives are picked from the filtered pool of sequences with optimal overall AG values (e.g., calculated using a package such as OligoWalk), or the change in Gibbs free energy associated with ASO:target binding corrected based on the ASO's propensity to dimerize or form hairpins.

The pipeline has produced gapmer sequences convergent with those designed by experts with therapeutic ASO design experience for two genes previously targeted, including sequences known to be successful in vitro. As a second validation step, the ASO pipeline returned splice modulating ASOs that overlapped with top candidates for a gene previously targeted with steric blocking chemistry. Finally, the pipeline successfully generated gapmers that downregulate the expression of a new key target gene associated with severe monogenic epilepsy, SCN8A, by >70% in a neuroblastoma cell line.

FIG. 3 shows success of the ASO design tools. 40 ASOs designed using the pipeline include candidates that reduce SCN8A mRNA levels by 70% and overlap with sequences recommended by an external expert.

The pipeline preferably includes at least certain sequence distance rules to avoid (2) off-target effects, such as excluding ASOs with exact matches, alignments with 1 mismatch, or alignments of at least 18 consecutive nucleotides to unintended transcripts for a 20 mer ASO. Despite the lack of appropriate transcriptome-wide data to support this claim, some experts in the field suggest that the modulation of unintended targets with more than this level of match is exceedingly rare. However, the invention includes the insight that those rules may be insufficient to fully exclude ASOs with unintended Watson-Crick binding and target modulation.

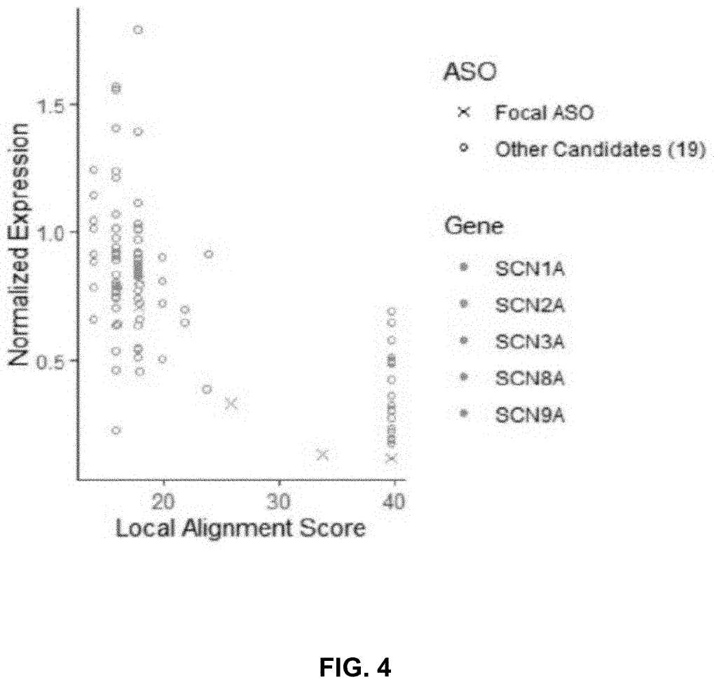

FIG. 4 shows modulation in vitro of unintended targets at greater sequence distance than those common thresholds. Importantly, the data show that the in vitro assays can give results predictive of CNS toxicity. SCN8A is one of a family of closely related sodium channel genes with a high degree of sequence similarity. To evaluate unintended modulation of other genes within this family by SCN8A-targeting ASOs, the levels of five sodium channel genes were measured using qPCR in neuroblastoma cells treated with 20 ASO candidates. Local pairwise alignments between the target sequences for these ASOs and the primary transcripts for the five genes were then performed using the Biostrings package in R. At least one ASO dramatically reduces not only the expression of its intended target, SCN8A, but also the expression of the related SCN3A and SCN1A transcripts, despite its alignments falling outside the thresholds used to exclude problematic off-target hits. The results show off-target modulation of closely related genes by SCN8A-targeting ASOs from expression of five sodium channels in neuroblastoma cells treated with one of 20 SCN8A-targeting ASOs. Local pairwise alignment (R/Biostrings) of the 20 mer target site of a focal SCN8A-targeting ASO to five sodium channel genes was performed.

The invention further uses the modeling the thermodynamics of interactions between the ASO and any potential binding partners may yield more conservative results than sequence distance rules and suggested ranges of appropriate changes in Gibbs free energy values to reduce the odds of off-target binding. The in silico operations may include a software module (e.g., RNAcofold) to predict the binding affinity of ASOs to sequences with 1-4 mismatches relative to their intended targets.

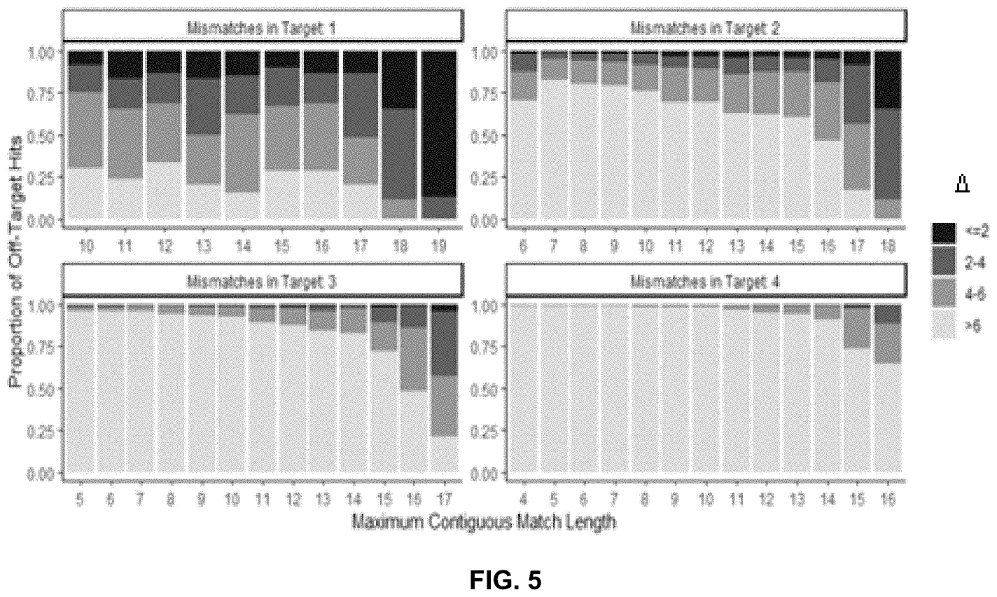

FIG. 5 shows predicted thermodynamic properties of ASO binding to mismatched targets (step 7). For a set of starting 20 mer ASOs (n=10), RNAcofold was used to predict AG, or the Gibbs free energy change (kcal/mol) associated with binding to fully complementary sequences and sequences with 1-4 mismatches. AAG categories, or the difference in AG between the ASO binding to the fully complementary and mismatched sequence, reflect relative affinity of the ASO to the intended and unintended targets. Standard exclusion rules rely on number of mismatches (panels) and stretch of alignment uninterrupted by mismatches (maximum contiguous match length, x-axis). For each “mutated” target sequence, AAG was calculated, or the difference in the Gibbs free energy of the ASO binding to the mutated target vs. binding to its direct 20 mer complement. This value was recently associated with a low probability of off-target modulation when >6 but relatively high probability of off-target modulation when ≤2. These results generally support the sequence distance heuristics used for off-target filtering in the design pipeline—the binding of an ASO to unintended targets with 1 mismatch or an 18 nt stretch of identity relative to an exact 20 mer match is likely to have a AAG value close to 0.

Once selected, the sequences are synthesized and exposed to live cells. The exposing step may be done by liquid handling systems of synthetic oligonucleotides into wells of multiwell plates that include the live cells, wherein the live cells are neurons, wherein at least one neuron expresses a microbial rhodopsin that functions as an optical reporter of membrane potential in the neuron.

The neurons may include Optopatch tools and provide a functional measurement platform. The exposure step provides an in vitro assay by which to record the electrical properties of neurons with an all-optical electrophysiology platform, Optopatch, which uses genetically encoded proteins for studies of the cell transmembrane potential.

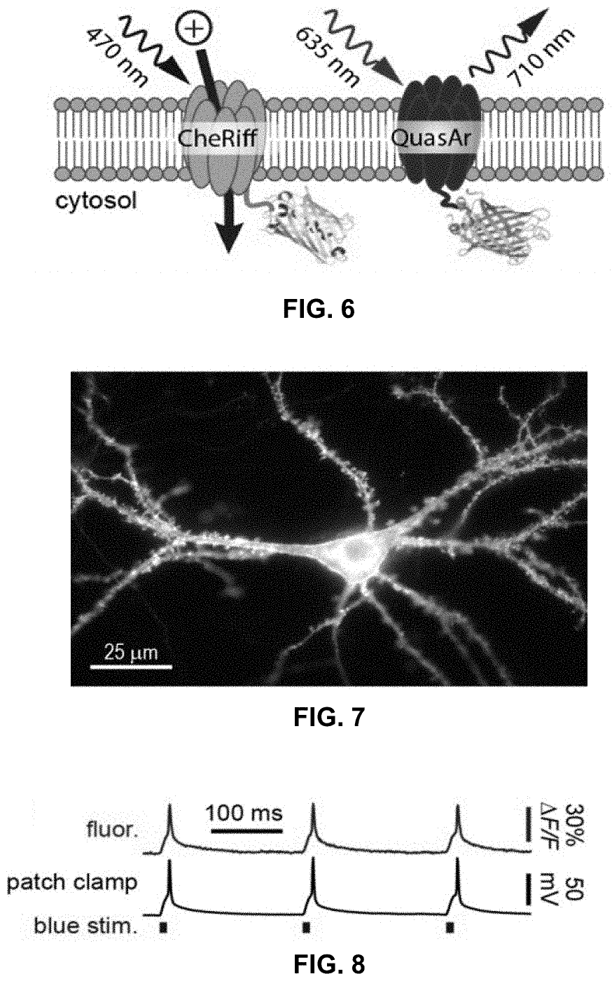

FIG. 6 illustrates an ASO being exposed to live cells with Optopatch. In preferred embodiments, blue light causes the channelrhodopsin protein CheRiff to open, triggering action potentials, while red light excites the fluorescent voltage sensor QuasAr to record the electrical activity of neurons.

FIG. 6 shows all-optical electrophysiology with Optopatch. Optopatch is comprised of two membrane proteins: a blue light-stimulated channelrhodopsin CheRiff and a red light-emitting voltage sensor QuasAr.

FIG. 7 shows membrane trafficking of CheRiff in a rat hippocampal neuron.

FIG. 8 shows QuasAr fluorescence faithfully tracks patch-clamp voltage recordings. Blue light triggers action potentials with high fidelity.

Those results are light that is emitted from neurons as those neurons fire action potentials. The 710 nm light emitted by QuasAr is emitted from positions along an axon where the action potential is traveling such that a movie of the neuron (see e.g., FIG. 7) shows light traveling along the length of the neuron. That light may be read and recorded as a movie, using, e.g., a microscope with a camera attached. The invention uses protocols for the routine delivery of genetic constructs expressing, e.g., the two Optopatch components into multiple excitable cell types, including rodent primary and human induced pluripotent stem cell-derived neurons. In addition, the invention uses a microscope for recording movies that store data of functional neuronal phenotypes (e.g., movies that show action potentials and firing patterns).

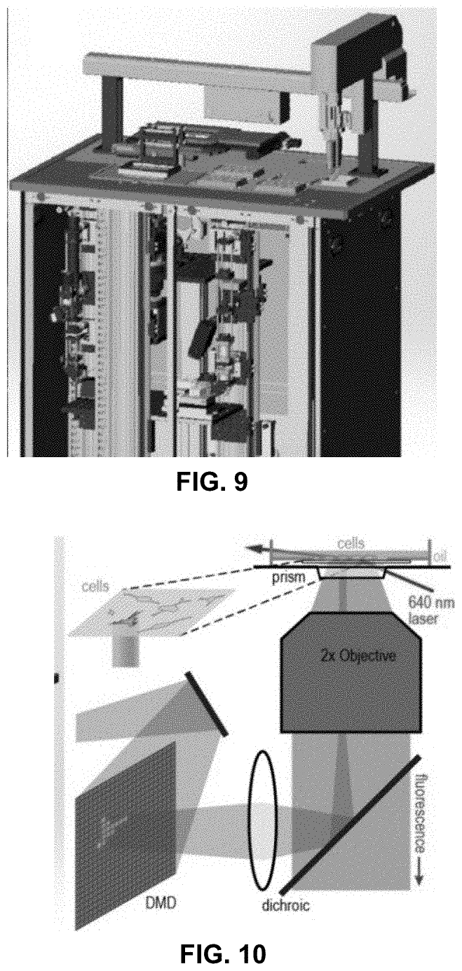

FIG. 9 shows a CAD model of a 96-well microscope for Optopatch measurements, aka an optogenetic microscope.

FIG. 10 shows the light path for coupling red laser light into cell samples via a prism for low-background voltage imaging, and the blue light path for patterned stimulation via a digital micromirror device (DMD) embodied within the optogenetic microscope. The optogenetic microscope provides for simultaneous voltage recordings from >200 individual neurons in a single movie, with 1 millisecond temporal resolution and single cell spatial resolution. The optogenetic microscope is a fully automated system compatible with 96-, 384- and greater well plates and can record from >600,000 neurons/day across 3000 wells/day, enabling high throughput screening campaigns with over 10,000-fold higher throughput compared to manual patch clamp electrophysiology. The cellular cultures in the optogenetics microscope preferably include human cell-based neuronal models and phenotypes. The cultures may include human induced pluripotent stem (iPS) cell-derived neurons, which may contain patient mutations in their native genetic and cellular context, through methodologies at scale for drug discovery applications. These systems provide in vitro measurements with clinical results for full assay validation. The approach serves as the basis for testing candidate therapies that ameliorate differences between cultured neurons derived from patient and control iPS cells.

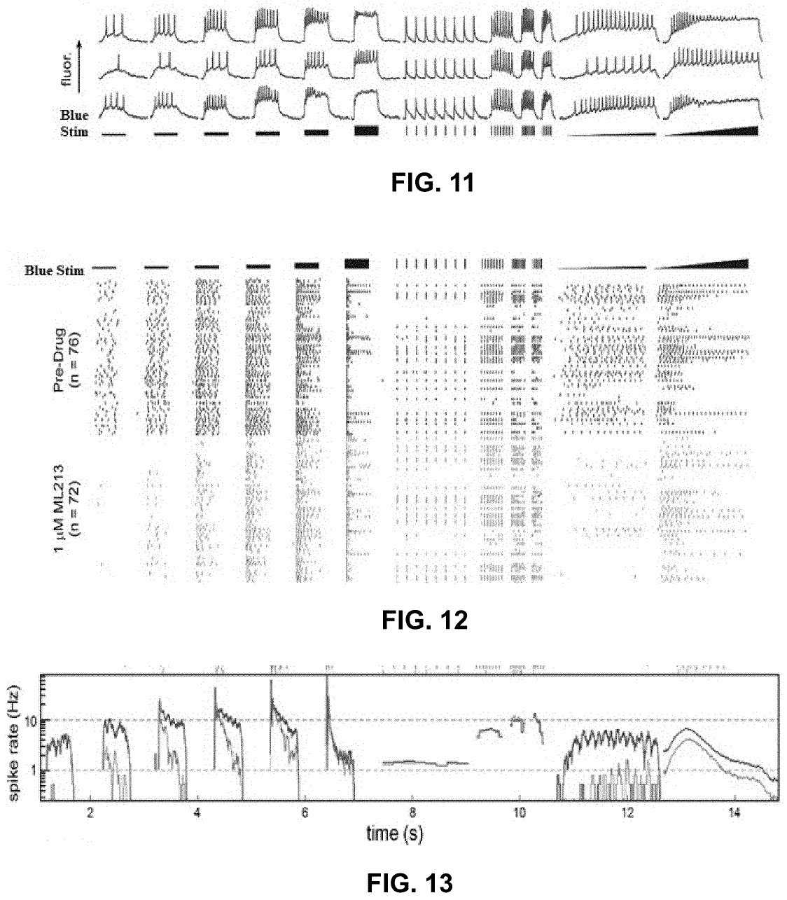

The invention uses robust Optopatch assays in multiple human iPS cell-derived neuronal types. FIG. 11 through FIG. 17 show the workflow for measurements of intrinsic excitability and pharmacological response in human iPS cell-derived motor neurons. Optopatch excitability in human iPS cell-derived motor neurons may be read from the optogenetic microscope with overlay (colored regions) of hiPSC-derived motor neurons identified by automated analysis.

FIG. 11 gives example voltage recordings from selected cells, and the blue stimulus used to evoke firing: steps, pulse trains, and ramps. The time scale across the bottom of FIG. 13 applies to FIGS. 11, 12, and 13.

FIG. 12 gives a Raster plot where each point is an identified action potential and each row is a neuron from a single FOV. Before (top) and after (bottom) addition at 1 μM of a test compound potassium channel opener that lowers resting potential and suppresses firing.

FIG. 13 shows the spike rate averaged over cells. The optogenetics microscope captures a variety of functional phenotype information in the form of firing frequency and action potential waveform, or spike shape, revealing the effects of composition on live cells.

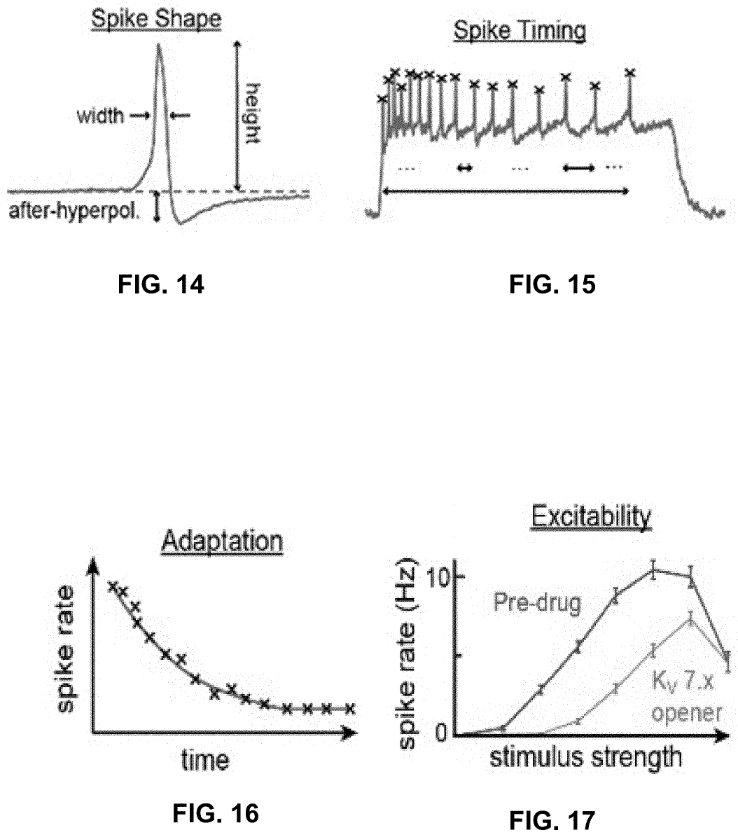

FIG. 14 shows spike shape recorded by the optogenetics microscope.

FIG. 15 gives spike timing properties (e.g., firing frequency).

FIG. 16 illustrates that adaptation are automatically extracted for each cell.

FIG. 17 shows the excitability extracted from the staircase in FIG. 13 shows a suppression in firing at all stimulus strengths.

Neurons are interrogated with a stimulus protocol (blue stim light) designed to probe a broad range of spiking behaviors. All pixels capturing fluorescence from one neuron co-vary in time following that cell's unique firing pattern. The temporal covariance is used to generate a weight mask for each cell; masked pixels are averaged for each frame in the movie to calculate the voltage traces. Each field of view (FOV) was recorded twice, before and after addition of the potassium channel opener ML213. Example traces in FIG. 11 demonstrate the underlying variability in neuronal behavior: recordings from many neurons must be averaged to reliably capture compound effects. From the fluorescence-time traces, each action potential in the dataset is identified (FIG. 12), and firing rate (FIG. 13), spike shape parameters (FIG. 14) and relative timings (FIGS. 15-16) are measured as a function of stimulus. The test compound clearly reduces neuronal excitability (FIG. 17. Around 500 parameters are automatically extracted by the parallelized analysis in the cloud and stored in a customized database. The extracted values, greatly reduced in number and complexity from the raw video data, serve as the substrate for more detailed analysis for distinguishing cell type, cell state, disease phenotype and pharmacological response.

Using the Optopatch platform and human cell-based assays, the invention is useful to measure the electrical properties of human iPS cell-derived neurons after treatment with ASO gapmers that targeted the downregulation of an ALS-linked gene.

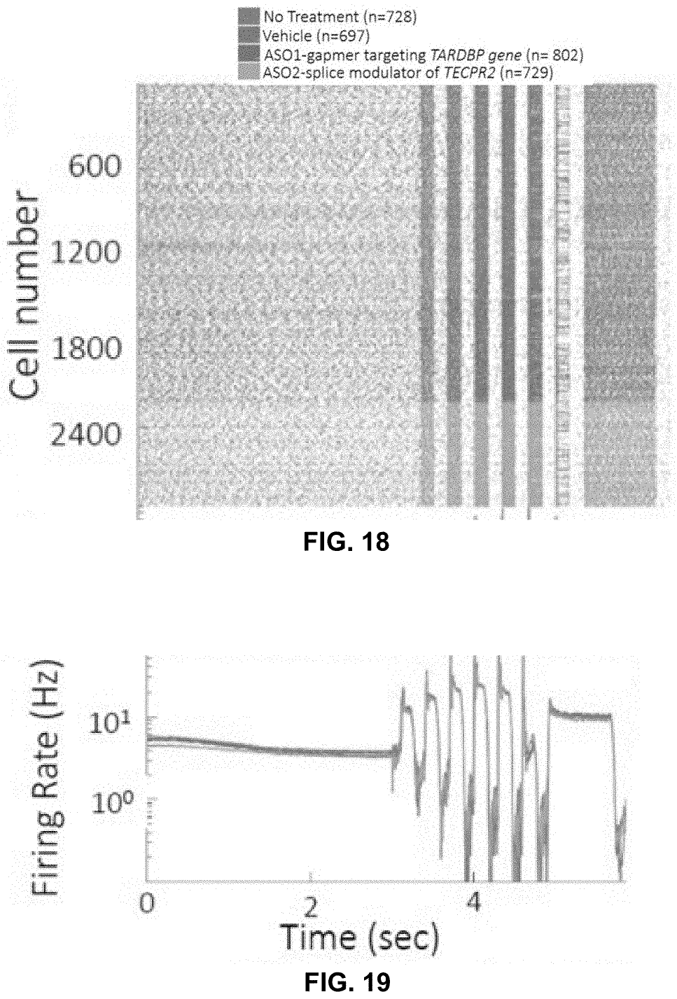

FIGS. 18-21 show Optopatch measurements of human iPSC-neurons treated with ASOs designed as gapmers to knock down the ASL-linked gene TARDBP (TDP-43), e.g., an “ALS-KD” embodiment. Those results show the ability to both modulate gene expression using ASOs in human cultured neurons and to carry out Optopatch measurements on the ASO-treated cells. Data was collected from a culture plate of human iPSC-neurons DIV30 (30 days in culture) treated for 20 days with an ASO gapmer targeting the downregulation of the ALS-linked gene TARDBP (encoding for TDP-43 protein) or a control ASO with RNA-like chemistry only and splice modulation activity against a different target (the TECPR2 gene). ASOs were delivered via transfection, and untreated and vehicle only conditions were also included as controls.

FIG. 18 is a Raster plot from the ALS-KD results, where each point is an identified action potential, and each row is a neuron. Note that the data was collected from ˜700-800 cells per condition for a total of 2956 single-cell Optopatch measurements.

FIG. 19 shows firing rate average in the ALS-KD embodiment for each condition over all cells measured for that condition.

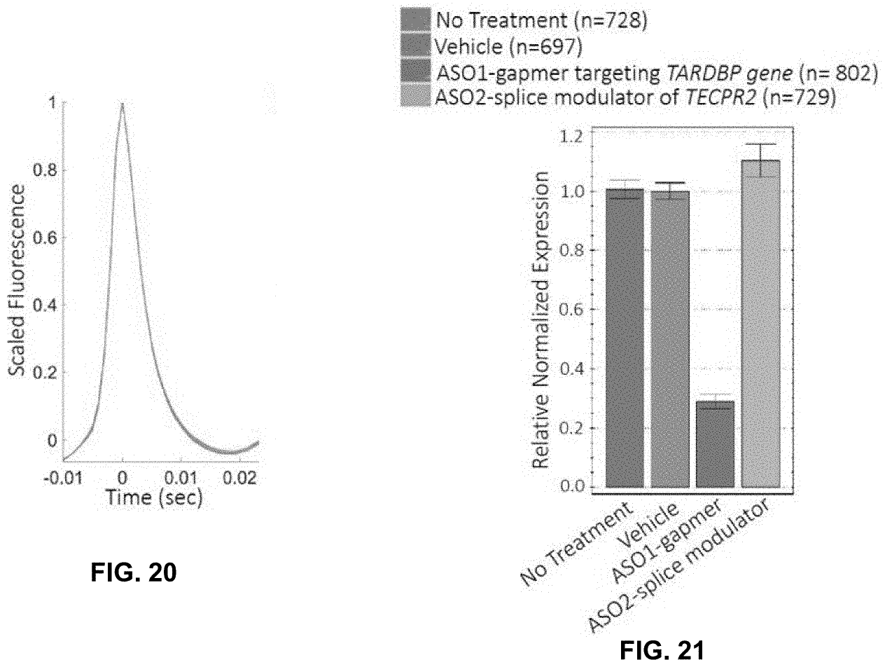

FIG. 20 is a spike waveform from the ALS-KD embodiment averaged for all cells measured per condition. The results showed no significant effect from vehicle treatment or any ASO treatment in the firing and spike waveform properties of the human iPSC-neurons measured.

FIG. 21 shows qPCR validation of ALS-KD target (TARDBP gene) downregulation in human iPSC-neurons treated with ASO1 gapmer, which showed ˜70% reduction in transcript levels (0.3 normalized expression) compared to control conditions.

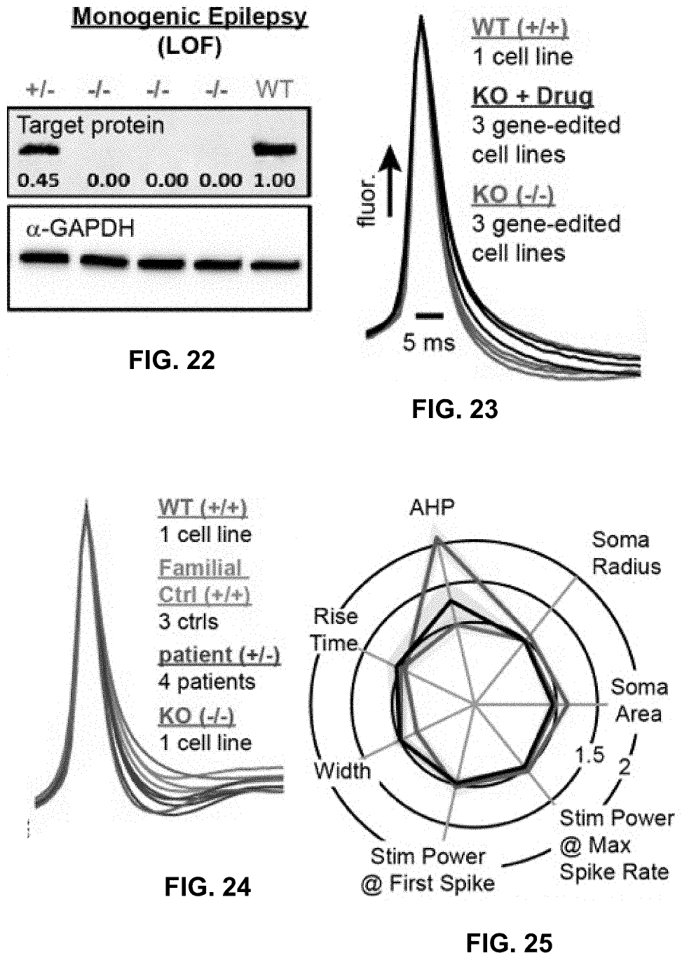

The invention uses in vitro screening and drug fingerprinting analytics to detect hallmarks of in vivo neurotoxicity. With a data store of the complex multi-dimensional measurements obtained with the Optopatch platform, the phenotyping framework combines machine learning techniques to identify uniquely discriminative sets of biological indicators, as well as inferential statistics to establish phenotypes that can be generalized across cell lines and experimental runs to arrive at a concise expression of disease effects articulated using real measures of electrophysiology. In screening efforts, the validated phenotypes may be reduced into composite parameters that can be used to rank and select compounds, and to characterize compound effects that change cell behavior in an off-target direction. The data store includes a phenotypic 30,000 compound screen from human iPSC-neurons (FIGS. 22-26), with examples centered around a disease model for a monogenic epilepsy.

FIGS. 22-26 show disease-associated phenotype identification & screening with Optopatch. The depicted embodiments in FIGS. 22-26 relate to monogenic epilepsy with an undisclosed mutation in iPSC-derived cortical excitatory neurons (NGN2 differentiation), cultured 30-45 days.

FIG. 22 shows results from WT cells, CRISPR/Cas9 was used to knock out the gene, and multiple isogenic clones were expanded and converted to neurons.

FIG. 23 shows through multiple rounds and KO cell lines, there was a consistent change in spike shape. Treatment with a clinically effective compound moves the behavior back towards WT.

FIG. 24 shows that a similar, but less severe phenotype was observed in heterozygous patient cell lines and healthy familial controls.

FIG. 25 gives a multidimensional radar plot that reveals changes in neuronal morphology, action potential shape, and spike train behavior. Treatment with the clinical compound moves the KO towards WT for all metrics. The radar plot transitions action potential data, recorded in movies from Optopatch, to quantitative features. A software module can collect action potential into radar plots and graduate the radial feature lines. Readings from those can be stored in as an input feature, e.g., as a vector. For example, the input feature may have a scalar value for each of AHP, soma radius, soma area, spike rate, first spike, width, and rise time. That vector (for each neuron under a given condition) may be handed to a machine learning system for analysis.

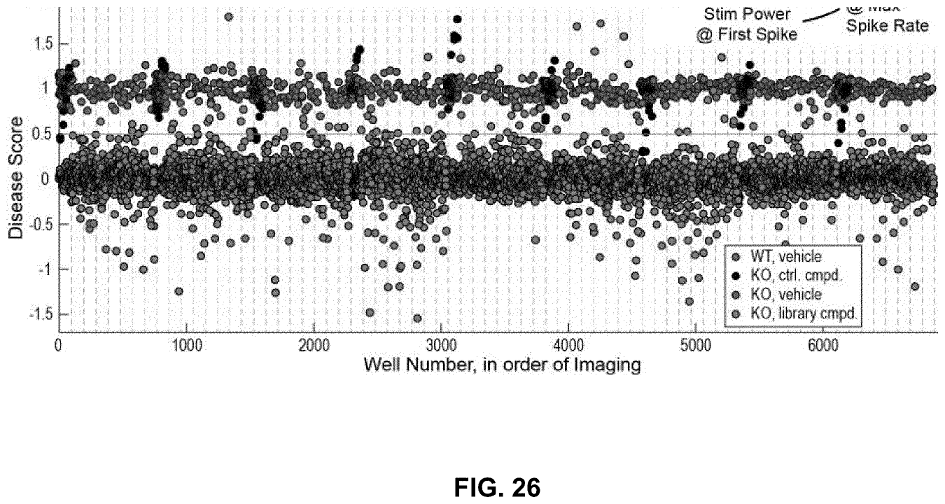

FIG. 26 shows that, for each cell culture, a multidimensional phenotype may be quantified by a disease score, a linear combination of many parameters, or features. A 30,000-compound screen was executed for molecules that reverse the phenotype; results from the first 6,900 wells are shown and have a ˜1.5% hit rate at a disease score threshold of 0.5. The results give a consistent phenotype and a stable assay (mean Z′=0.4). Each dashed gray line corresponds to one 96-well plate.

This work shows the ability to obtain an Optopatch phenotype that is consistent across multiple CRISPR/Cas9-edited and patient-derived cell lines (FIG. 23, 24). The phenotype can be nearly entirely reversed by a drug approved for the condition, showing important validation of the platform methodology of extracting disease-relevant functional parameters (FIG. 23-25). This Optopatch phenotypic screen (run on billions of high-quality iPSC-neurons) showed excellent assay consistency in cultured human neuron across multiple plates measured in different weeks with a large screening window and clearly identified hits, underscoring the ability to execute and analyze complex assays with human iPS cell-derived neurons.

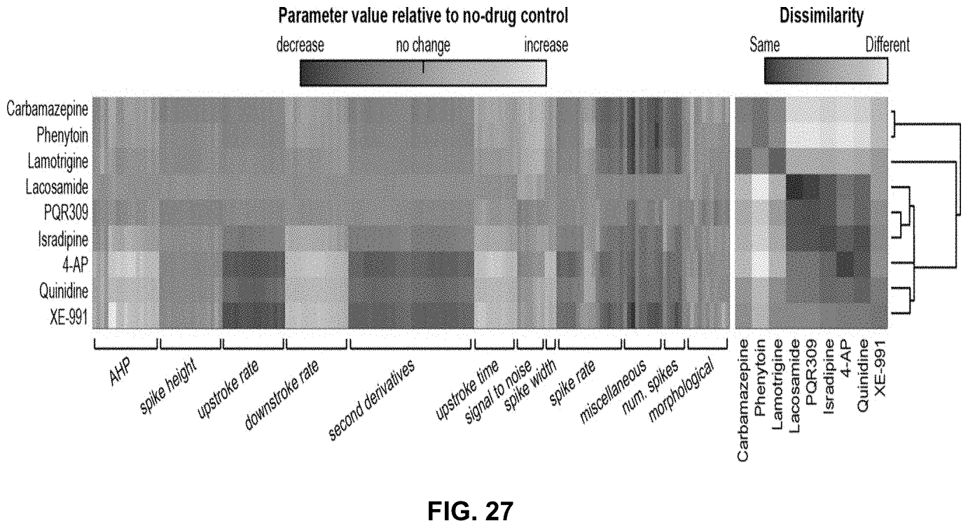

The invention includes deep-learning techniques for exploiting the “fingerprints” of intervention effects that may be observed when a therapeutic candidate (either small molecule compounds or ASOs) is included in a screening campaign, capturing “neighborhoods” of interventions with similar functional effects (FIG. 27).

FIG. 27 shows drug similarity comparisons. Using input features (e.g., vectors of features from Optopatch) a machine learning system may identify that a new drug has a similar fingerprint to a known drug in the data store. In some embodiments, hiPSC neurons were cultured 30 days and treated with tool compounds to build the data store. Parameter values relative to no-drug control shows the drug response captured by 162 parameters extracted from the Optopatch excitability assay. Different parameters within a category correspond to different stimulus regimes. Sodium channel blockers and potassium channel blockers behave similarly to each other. A dissimilarity matrix shows neural measurements for each compound reduced to a concise fingerprint via manifold learning techniques, then mapped into neighborhoods by fingerprint similarity in the reduced drug space. Here, a dissimilarity matrix is shown comparing two independent rounds of measurement, showing repeatability of compound fingerprints and clustering of similar compounds. Such results make up a data store, a reference library of drug fingerprints from tool compounds and approved drugs for use as landmarks in the space of electrophysiology explored by assays of the disclosure. The data store can be used in in vitro screening efforts (by mapping a disease phenotype onto the compound space to find relevant compound neighborhoods) and/or provide a hot-start for target deconvolution efforts. In preferred embodiments, the analysis is performed by a machine learning system trained on the data store, wherein phenotypes in the data store are associated with condition labels.

All these tools are useful for predicting in vivo neurotoxicity with in vitro neurophysiology read-outs. The in vitro tests use human cellular model generation, high-throughput functional electrophysiology with Optopatch, and machine learning analytics to identify predictive electrophysiological signatures of ASOs likely to produce neurotoxicity side effects in vivo. The multidimensional readouts are useful to identify the impact of perturbative ASO activity in the context of in vivo relevant neuronal cell types.

Systems and methods of the disclosure are useful to design ASOs that modulate the level of therapeutically relevant RNAs in neurons. Optogenetics and machine learning are used to predict and avoid ASO toxicity in vivo in a platform that integrates neuronal-based disease models, high-throughput all-optical electrophysiology (or Optopatch), and machine-learning based analytics. Methods include in silico operations that systematically characterize the sequence and thermodynamic rules that govern ASO modulation of unintended RNA targets. Methods include in vitro assays that predict in vivo toxic neurological phenotypes using the Optopatch platform. Results are included here that show components of the systems and methods wherein ASOs have been designed and evaluated to modulate genetic targets.

To summarize, methods include systematically characterizing the relationship between sequence distance and off-target ASO activity for both gapmers and steric blocking oligonucleotides. Sequence complementarity to an intended target and sequence distance (or number of mismatches) relative to unintended targets drive ASO specificity, but the distance thresholds that determine interactions with unintended targets are improved here. Systems and methods of the invention consider that target site accessibility (resulting from RNA secondary structure or RNA binding protein occupancy) for determining both an ASO's on-target efficacy and its ability to modulate off-target binding sites. The observation that an ASO does not modulate a close sequence match may result from inaccessibility of that site on the transcript rather than its inability to bind to that semi-complementary sequence in isolation, which could lead to sequence heuristics that are inappropriately permissive in the absence of target secondary structure or protein-binding. In order to probe the relationship between sequence distance, thermodynamics, and ASO efficacy, target site accessibility must be accounted for.

Systems and methods of the disclosure my provide ASOs that embody clinically relevant ASO chemistries: 2′-MOE containing “gapmers” that direct the RNase H-mediated decay of their targets and “steric blocking oligonucleotides” which may modulate transcript splicing, stability, or translation. A relationship between AAG (the difference in duplex formation energy between the ASO binding to a mismatched target vs a fully complementary target) and off-target modulation of transcript level may be modeled and predicted in silico and used to guide the design of the panels of test oligonucleotides. Preferably ASOs will be designed to span a range of sequence distances (1-4), stretches of contiguous binding, mismatches in preferred RNase H cut sites, and AAG values. ASO delivery and readout assays are used to assess the efficacy of those hundreds of candidate ASOs. Results may be fed back into the informatics pipeline to generate conservative rules to exclude ASOs with off-target effects while accounting for accessibility at the target site.

Certain embodiments use bulk RNA-sequencing to evaluate transcriptome-wide gene expression and isoform composition in cultured human neurons treated with a panel of informative ASOs to identify off-target activity at various levels of sequence similarity. Those experiments provide for the characterization of off-target effects on both transcript levels and splice modulation for the two most clinically relevant ASO chemistries. The tests may include untreated cells, cells treated with transfection reagent/vehicle, cells treated with non-targeting ASOs of both chemistries as negative controls, cells treated with at least one positive control ASO from the literature with known off-target effects, and candidate ASOs designed by methods of the invention. For each target, existing RNA-Seq tools may be used to perform differential expression analysis for the paired ASOs known to modulate the same intended target and the negative control conditions. Genes whose expression is significantly modulated by both ASOs targeting a gene relative to negative controls are likely to be secondary effects of the intended target knockdown, while genes that are significantly modulated by only one of the two ASOs are likely to be the result of Watson-Crick binding to unintended target sequences. A combination of sequence analysis and thermodynamic modeling may be used to predict off-target genes likely to be modulated by each of the ASOs. Overlap between predicted off-target hits and observed off-target modulation of gene expression may be used to generate refined exclusion criteria for in silico off-target filtering. Genes which are likely to be direct effects of a particular ASO but are not initially identified by our standard sequence analysis may be especially valuable in refining the design rules.

Further systems and methods of the disclosure are used to demonstrate that in vitro functional measurements of neurons can predict in vivo CNS toxicity. An in vitro assay predictive of in vivo neurotoxicity allows ASOs with neurotoxic liabilities to be removed from the drug development pipeline prior to expensive and labor-intensive in vivo work in rodents and non-human primates. Here, exposing the live cells to the oligonucleotides in vitro preferably includes performing all-optical electrophysiology or Optopatch to obtain a neural phenotype for the cells when exposed to the oligonucleotides. Optopatch may be used to characterize the behavior of primate or rodent primary and human iPS cell-derived neurons at high-throughput. Optopatch provides a valuable in vitro screening tool for this purpose for several key reasons. First, Optopatch assays demonstrably reflect the functional effects of both genetic lesions (FIGS. 22-25) and pharmacological interventions (FIGS. 11-13 and 27). Here, relevant neurotoxic mechanisms may be reflected in these functional measurements. Methods of the invention may use a wealth of historical Optopatch data (a data store) characterizing the effects of, e.g., FDA-approved small molecule compounds, allowing ASO-driven Optopatch phenotypes to be put within a wider context of effects that are (or are not) well-tolerated in the clinic.

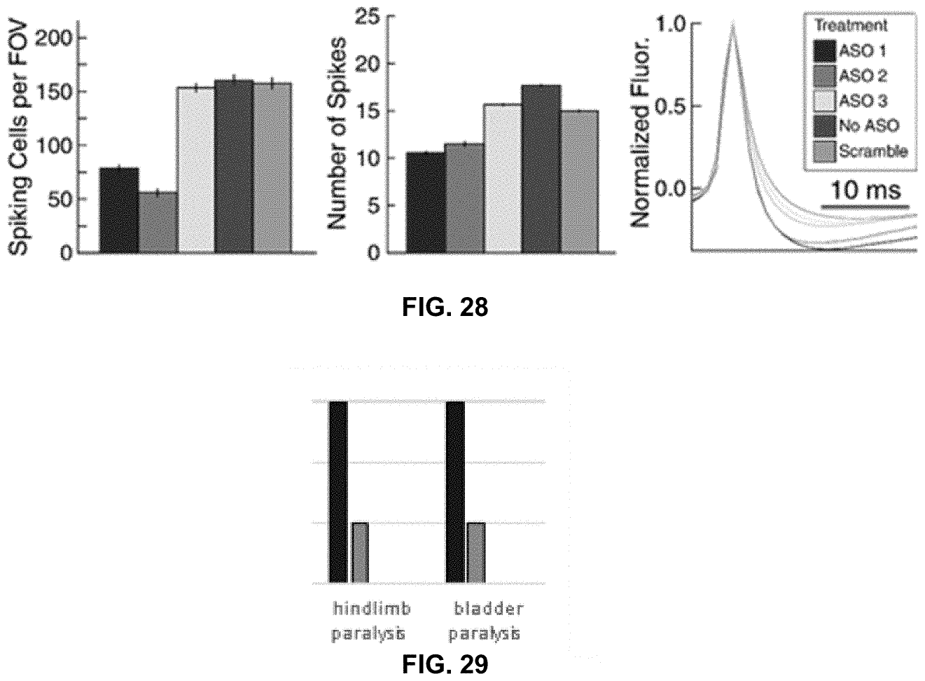

A pilot characterization of 3 gapmer ASO candidates suggests that chronic neurotoxicity is reflected by Optopatch phenotypes. ASO1 and ASO2, but not ASO3, altered the functional phenotypes of cultured rat hippocampal neurons (DIVE) 7 days after ASO delivery (FIGS. 28-29). The results show that Optopatch phenotypes correlated with in vivo neurotoxicity. ASO 1, ASO 2, and ASO 3 have distinct sequences but reduce the expression of the same target gene.

FIG. 28 show results from rat hippocampal cultures treated with ASO 1 (n=1,413 neurons), ASO 2 (n=973), ASO 3 (n=2,761), no ASO (n=2,880), and a scrambled control ASO (n=2,836). Cultures treated with ASO 1 and ASO 2 have fewer spiking cells per field of view (FOV), fire action potentials less frequently, and have altered action potential waveforms relative to cultures treated with ASO 3 or control compounds.

FIG. 29 show results when ASO 1, ASO 2, ASO 3, and vehicle were intrathecally delivered to rats. No toxic phenotypes were noted at 24 hrs post injection for any compounds, but bladder and hindlimb paralysis 10-12 days post injection were noted for animals treated with ASO 1 and ASO 2. Darkness, or gray levels, match FIG. 28.

While none of the ASOs produced neurological phenotypes in vivo in rats 24 hrs after IT delivery, hindlimb and bladder paralysis at days 10-12 after IT delivery were observed in rats treated with ASO1 and ASO2 but not in those treated with ASO3 (FIG. 29).

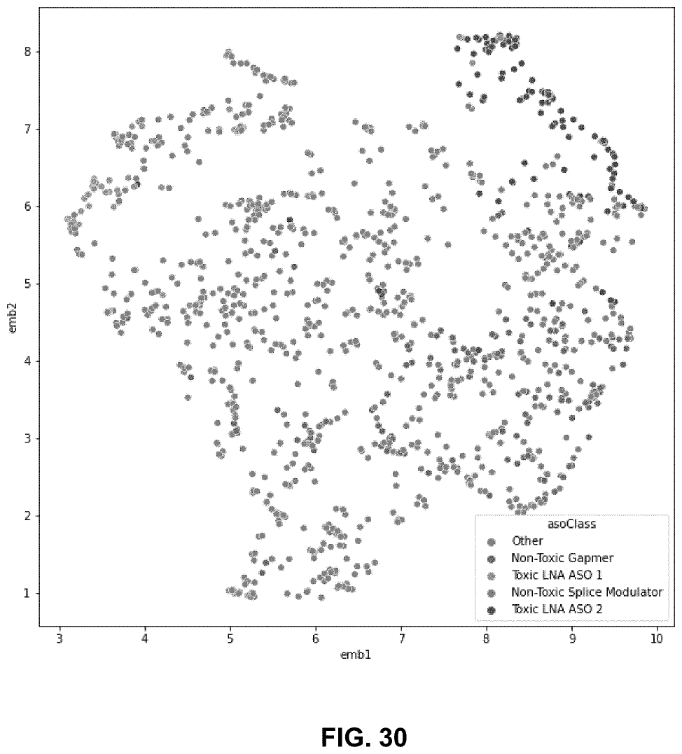

In FIG. 30, a UMAP projection based on 602 based on features of intrinsic excitability separates two ASOs known to be toxic in vivo from two ASOs known to be tolerated in vivo. Additional data points plotted in this space show the behavior of neurons treated with ASOs targeting a therapeutically relevant mRNA. These data can be used to de-prioritize sequences that resemble toxic ASOs in the in vitro assay. Given those promising pilot data, the evidence suggest that methods of the invention provide an in vitro screening assay to predict ASO-driven neurotoxicity and efficiently eliminate neurotoxic ASO candidates early in lead discovery.

### EXAMPLES

The experiments of the examples validate that in vivo neurotoxic ASO effects can be predicted by aberrant patterns of neuronal activity in two useful cellular models.

### Example 1.1

Generate a panel of 400 diverse ASOs representing the two most clinically relevant chemistries and characterize their effects on functional behavior and cytotoxicity in rodent primary neurons. This panel will include a series of 10-20 positive control ASOs known to be toxic in vivo, including oligonucleotides known to induce acute seizures in mice, those with known hepatotoxicity, and representatives that cause hindlimb weakness or other neurotoxic phenotypes in vivo. The panel will also include a series of 10-20 negative control ASOs that have progressed to the clinic and are known to be well tolerated in vivo in humans and in model species used for preclinical toxicology. The remaining ASOs will be non-targeting (within the thresholds established in Aim 1) but this set will contain a broad spectrum of sequence motifs and will be generally representative of the sequences selected during our normal design process (e.g., have moderate GC content, avoid CpG motifs, etc.). Those sequences will be synthesized using both gapmer and steric blocking chemistries to evaluate the interaction between chemistry and sequence and its effect on likely toxicity. The behavior of cultured rat cortical and dorsal root ganglion neurons treated with this panel of ASOs will be functionally characterized using Optopatch. Functional characteristics that separate non-targeting and positive control ASOs from ASOs known to be well-tolerated clinically will be identified, and these characteristics will be used to select subsets of the non-targeting ASO panel that are most (n=10) and least (n=10) likely to have neurotoxic phenotypes in vivo.

### Example 1.2

Characterize the functional behavior and cytotoxicity profiles of different types of human iPS cell-derived neurons treated with the panel of oligonucleotides tested in Example 1.1. The also be tested in human iPS cell-derived neurons to establish whether any of the functional effects are species-specific. When this is the case, human iPS cell-derived neurons may be a more faithful model to predict clinical toxicity than rodent models. Use at least two different types of human iPS cell-derived neurons, cortical excitatory “NGN2” neurons produced in-house by transcriptional programming and commercially available differentiated neurons (iCell Neurons produced) by Cellular Dynamics International. NGN2 neurons will be generated by driving the overexpression of the pro-neuronal transcription factor Neurogenin 2 in genetically modified iPS cell lines. iPS cell-derived neurons will be co-cultured with primary mouse glia monolayers to allow for maturation. ASOs will be delivered using established protocols.

### Example 2

Characterize the 20 ASOs identified in Example 1.1 in vivo by intrathecal delivery in rats. Each ASO will be tested in 5 rats and the animals will be observed for 2 weeks to evaluate both acute and longer-term neurotoxic effects. Initial work will utilize only male rats. As it is unlikely that off-target homology-based binding effects and general neurotoxic effects will be sex-specific, the intent is to reduce variability and improve power to detect ASO-mediated effects in these initial experiments by focusing first on male animals (Robustness and Reproducibility). Any major findings will be confirmed in female animals as part of future studies.

### Example 3

Test refined ASO design criteria and improved screening tools on a therapeutically relevant target gene. Design and test ASOs targeting UBE3A, a gene associated with two distinct neurodevelopmental disorders—Angelman syndrome and Chromosome 15q duplication syndrome (Dup15q). This example uses the disclosure of U.S. Provisional Application 63/150,188, filed Feb. 17, 2021, incorporated by reference. Angelman syndrome results from loss-of-function mutations and deletions of the maternal copy of UBE3A, whereas duplications or triplications of the chromosomal region harboring maternal UBE3A lead to Dup15q. The maternal nature of mutations is due to genomic imprinting of the UBE3A gene in a neuron-specific manner, whereby a long non-coding antisense transcript silences the paternal copy of UBE3A in neurons only. Although clinically distinct, both syndromes present with seizures, motor impairments, language delays/impairments, and often meet the criteria for autism spectrum disorder. UBE3A encodes an E3 ubiquitin ligase protein that acts in the cytoplasm and nucleus and targets substrates for degradation by the proteosome. Although the exact targets of UBE3A are still being investigated, synaptic dysfunction and changes in neuronal intrinsic excitability are associated with deletions and duplications of UBE3A, highlighting its critical function in neurons and normal brain physiology. ASOs represent an attractive therapeutic approach for disorders associated with UBE3A. Targeting the paternal antisense transcript has already been explored for “un-silencing” the paternal copy of UBE3A to restore function in Angelman syndrome. For Dup15q Syndrome, knocking down the increased UBE3A levels to wild type levels with a gapmer ASO may help to rescue these phenotypes and provide therapeutic benefit to affected patients.

Here, ASOs which utilize gapmer chemistry to induce RNaseH-mediated decay of excess UBE3A transcript in Dup15q syndrome are used. The experiments outlined below will test the ability to avoid toxicity when designing and screening ASOs.

### Example 3.1

Design 40 gapmer ASOs to down-regulate UBE3A expression using updated thresholds for off-target exclusion. See e.g., 63/150,188, filed Feb. 17, 2021, incorporated reference. Evaluate on-target gene modulation in patient-derived fibroblast and iPSC-neurons using qPCR and protein levels for all ASOs to identify candidates that appropriately modulate transcript and protein levels of UBE3A. Use Dup15q dermal fibroblast cell lines obtained from Coriell and patient iPS cell lines and isogenic controls. iPS cell lines will be differentiated into cortical excitatory neurons using a robust transcriptional programming approach mediated by overexpression of the proneuronal transcription factor NGN2.

FIG. 22-25 generally show results of such methods, wadapted to facilitate screening efforts. At least three independent rounds of ASO delivery and readout assays (qPCR and immunoblotting) will be carried out in each cell type to assess the efficacy (percentage of UBE3A transcript and protein knockdown) of the molecules designed using the refined criteria. For the differentiated human neurons, use established ASO delivery conditions compatible with downstream measurements (e.g., FIG. 11-13). Test different timepoints for ASO delivery and duration of treatment to further understand the dynamics of UBE3A expression and modulation in these cells.

### Example 3.2

Screen all 40 candidates using Optopatch in primary rodent neurons and human iPS cell-derived neurons. Functional characteristics of primary rodent neurons treated with the 40 ASO candidates will allow us to use the predictive neurotoxicity thresholds developed in Example 1.1 to flag and exclude candidates likely to cause toxic effects in vivo.

In addition, results from screening in human iPS cell-derived neurons are useful to evaluate these phenotypes in the context of the ASO candidate's therapeutic window. The human iPS cell-derived neurons tested here will include control cells with normal expression of UBE3A as well as neurons differentiated from Dup15q patient iPSC lines. Use at least one set of isogenic cell lines in which the Dup15q duplication has been genetically corrected. Cortical excitatory neurons differentiated from these cell lines will be co-cultured with primary mouse glia for maturation and genetic constructs encoding Optopatch components will be delivered via lentiviral transduction two weeks prior to all-optical electrophysiological measurements. Initial rounds of Optopatch measurements will focus on assessing phenotypic differences between the patient and control neurons. The Optopatch platform will likely identify several neuronal excitability phenotypes associated with the disease condition. Having established a phenotype, assess the effect of the 40 ASOs on the Optopatch parameters of control and Dup15q neurons.

The use of multiple cell lines representing the patient and control genotypes will allow us to quantify both on-target functional rescue and any off-target signatures of toxicity. Dose-response studies will be useful to identify ASO concentrations that are therapeutically relevant but avoid toxic phenotypes. Those data will demonstrate the power of the Optopatch system to both qualify ASO candidates for desired phenotypic rescue and eliminate those with undesirable perturbative effects on neuronal activity.

### Example 3.3

Perform RNA-seq on human iPS cell-derived neurons treated with the top 3 candidate ASOs and vehicle controls. Genes that are differentially expressed between neurons treated with candidate ASOs (n=5 replicates per ASO) targeting UBE3A will be mined to identify potential off-target Watson-Crick binding. This will be useful to identify off-target transcript modulation. The functional relevance of any differential expression observed in ASO-treated neurons relative to vehicle-treated ASOs (n=5) may be assessed using Optopatch phenotypes.

### Example 3.4

Test the top 3 candidate ASOs for tolerability in vivo in rats. Each ASO will be intrathecally delivered to 5 rats and clinical examinations of these animals in the 2 weeks following ASO treatment will be scored relative to vehicle-treated animals (n=5). This will serve as a test of the toxicity predictions. Optopatch phenotypes in primary rodent neurons will be useful to exclude ASOs that induce neurotoxic effects in vivo.

To summarize, systems and methods of the disclosure are useful for predicting and avoiding off-target effects and neurotoxicity in ASO design.

1. A method comprising:
generating a list of oligonucleotide sequences that are substantially complementary to a genetic target implicated in a disorder;
analyzing the sequences via in silico operations that remove sequences from the list according to pre-determined criteria, leaving a filtered list;
obtaining oligonucleotides made with sequences from the filtered list; and
exposing one or more live cells to the oligonucleotides in vitro to identify candidate therapeutic oligonucleotides that do not induce an adverse phenotype in the live cells.

2-3. (canceled)

4. The method of claim 1, wherein the in silico operations include comparing each oligonucleotide sequence to a genome and removing ones that are substantially complementary to a sub-sequence in the genome outside of the genetic target.

5. The method of claim 1, wherein the in silico operations include removing sequences from the list for which binding affinity to its intended target is insufficiently favorable.

6. The method of claim 5, wherein the in silico operations include a software module that models duplex formation and associated Gibbs free energy changes to exclude sequences that: form dimers, form hairpins, or bind off-target.

7. The method of claim 1, wherein the in silico operations include comparing the list of oligonucleotide sequences or the genetic target to a genome of a non-human model organism to identify a genetic target with homologous target in the non-human model organism.

8. (canceled)

9. The method of claim 1, wherein the live cells comprise stem-cell derived neurons in vitro.

10. The method of claim 9, wherein at least one of the neurons comprises an optical reporter of membrane potential, and the method includes using a light detector or sensor to read a neural activity phenotype of the neuron when exposed at least one of the oligonucleotides.

11. The method of claim 10, wherein the neurons include a light-gated ion channel.

12. The method of claim 9, wherein the neural activity phenotype is analyzed against a data store of phenotypes.

13. The method of claim 12, wherein the analysis is performed by a machine learning system trained on the data store, wherein phenotypes in the data store are associated with condition labels.

14. The method of claim 13, wherein the phenotypes in the data store are labeled by neurological conditions that include one or more of epilepsy, autism, and Alzheimer's disease.

15. (canceled)

16. The method of claim 1, wherein the in silico operations include predicting the performance of the oligonucleotide sequences as gapmers that will mediate enzymatic degradation of an RNA.

17. The method of claim 16, wherein the genetic target is a gene for a sodium channel.

18-19. (canceled)

20. The method of claim 1, wherein the in silico operations include predicting the performance of the oligonucleotide sequences as splice-modulating oligonucleotides that promote splicing of a pre-RNA to form a preferred isoform of an RNA.

21. The method of claim 1, wherein the in silico operations include predicting the performance of the oligonucleotide sequences as steric blocking oligonucleotides that inhibit the function of a micro-RNA.

22. The method of claim 1, wherein the in silico operations include presenting the oligonucleotide sequences to a predictive module that predicts target-binding by comparison to results from transcriptomic analysis assays performed with test oligonucleotides.

23. The method of claim 22, wherein the predictive module uses a machine learning system to predict expression modulation of off-target genes for each oligonucleotide sequence, the machine learning system trained on results of expression analysis for a plurality of antisense oligonucleotides.

24. The method of claim 1, wherein the in silico operations include the application of sequence distance rules to avoid off-target effects, wherein the rules exclude sequences for which the genome includes a non-target region that aligns to the sequence with an exact match, 1 mismatch, or at least a threshold number of consecutive matches.

25. The method of claim 1, wherein the in silico operations include software packages that perform a pairwise alignment of each of the oligonucleotide sequences to a human genome or to a primary transcript sequence for a gene that includes the genetic target to exclude sequences with off-target binding affinity.

26-27. (canceled)

28. The method of claim 1, wherein the in silico operations include evaluating, for each oligonucleotide sequence, accessibility of a binding site in the genetic target wherein accessibility is evaluated by a software module that predicts secondary structure or binding protein occupancy in an RNA transcript of the genetic target.

29-43. (canceled)
